(19) United States
(12) Patent Application Publication
(10) Pub. No.: US 2022/0047385 A1
(43) Pub. Date: Feb. 17, 2022

(54) Electrospun polymer assemblies for medical implant applications

(71) Applicant: Xeltis AG, Zurich (CH)

(72) Inventors: Yonatan Gray, Paris (FR); Martijn Antonius Johannes Cox, Budel (NL)

(21) Appl. No.: 17/437,230

(22) PCT Filed: Mar. 11, 2020
(86) PCT No.: PCT/EP2020/056467

§ 371 Date: Sep. 8, 2021

Related U.S. Application Data
(60) Provisional application No. 62/816,533, filed on Mar. 11, 2019.

(51) Int. Cl. A61F 2/24

(52) U.S. Cl. CPC A61F 2/2418; A61F 2240/001; A61F 2220/005; A61F 2220/0058

(57) ABSTRACT

A medical implant is provided that has a first and a second electrospun component with the same type of biodegradable electrospun polymers. In one example, the second electrospun component is separately manufactured from the first electrospun component. Furthermore, the implant is structured such that the first electrospun component and the second electrospun component are assembled or joint together by the same type biodegradable electrospun polymers as in the first electrospun component and the second electrospun component. The assembled implant is a porous, biodegradable medical implant capable of being replaced by naturally ingrown tissue over time upon implantation. Advantages are the avoidance of sutures and the problems associated with the use of sutures, capability of ETR, avoidance of the need for extra materials, allowance for more precise and reproducible assembled structures for which the process could be automated.

### FIELD OF THE INVENTION

This invention relates to tissue engineering products and methods.

### BACKGROUND OF THE INVENTION

Cardiovascular diseases are one of the biggest causes of deaths worldwide. One way to treat at least some of these diseases is by tissue engineering. Tissue engineering can be used for the replacement of cardiovascular tissues, such as arteries and heart valves. Most commonly used heart valve replacements today are bioprosthetic heart valves, which typically contain mainly animal-derived tissue as leaflet material. The tissue of the leaflets is normally sewn together and connected to the valve by stitching. Moreover, the animal tissue is going through chemical fixation to allow specific shape set for optimal hemodynamics without compromising material properties and leaflet durability.

Currently used cardiovascular substitutes encounter risks due to coagulation, infections, degeneration, and no growth possibilities. Tissue engineering is based on endogenous tissue regeneration (ETR) and uses patient's own cells and a biodegradable polymer scaffold to make autologous tissue that is able to grow, adapt and repair. To ensure proper cell and tissue growth, the scaffolds must be highly porous and match the mechanical properties of the tissue.

Electrospinning is a technique that produces polymer nanofibers using a high voltage electrostatic field. It results in a highly porous material of nanofibers that resembles the extra cellular matrix of the tissue. Tissue engineering can, for example, be used for coronary bypass grafts, heart valve replacements, AV shunts for dialysis patients.

When considering bioabsorbable polymeric scaffolds as leaflet material, common methods are not always applicable and stitching techniques demonstrate compromised durability due to tearing especially in areas of higher stress where the stitches are located.

The bioprosthesis surgical aortic valve geometry and shape in diastolic phase dictates a stress concentration at the higher part of the posts. When the valve is fully loaded, the posts will deflect inwards and due to stiffness difference between the rigid metallic frame and the flexible leaflet material, high shear stresses will conform at the upmost part of the attachment surface.

Another aspect to consider as a disadvantage for a use of stitches at the posts would be a possible formation of calcification deposits around the posts in vivo which is caused by stitches and/or laser cut holes for accurate sewing of the stitches. As the posts are stagnant relatively to the very dynamic leaflet, enhanced tissue formation is expected to initiate there. It has been observed to some extent that calcification deposits show up around the posts at specific stitches locations. This observation could potentially lead to degraded valve hemodynamics and in vivo durability.

The present invention addresses these problems and advances the art by providing medical implants based on other techniques than stitching.

### SUMMARY OF THE INVENTION

The present invention provides a cardiovascular medical implant or medical implant that has a first electrospun component with a type of biodegradable electrospun polymers and a second electrospun component with the same type of biodegradable electrospun polymers as in the first electrospun component. In one embodiment the second electrospun component is separately manufactured from the first electrospun component. Furthermore, the cardiovascular medical implant or medical implant is structured such that the first electrospun component and the second electrospun component are assembled or joint together by the same type biodegradable electrospun polymers as in the first electrospun component and the second electrospun component. The assembled cardiovascular medical implant or medical implant is a porous, biodegradable medical implant capable of being replaced by naturally ingrown tissue over time upon implantation. The assembly or joint assembling the first electrospun component and the second electrospun component together is an ultrasonic weld or is a glued weld. Examples of assembled cardiovascular medical implants are where the first electrospun component and the second electrospun component are components of a tissue engineered heart valve, a tissue engineered vascular graft, or a tissue engineered vessel.

In one embodiment, the cardiovascular medical implant or medical implant is structured such that the first electrospun component and the second electrospun component are not sewn or stitched together, or such that the first electrospun component and the second electrospun component do not have any sewn areas or stitches.

In a further embodiment, the cardiovascular medical implant or medical implant has a support structure assembled or joint together in between the first electrospun component and the second electrospun component.

The present invention further provides a tissue engineered heart valve that has two (or more) separately manufactured electrospun heart valve components, each manufactured with the same type of biodegradable electrospun polymers. The two separately manufactured electrospun heart valve components are assembled or joint together by the same type biodegradable electrospun polymers such that the assembled tissue engineered heart valve is a porous, biodegradable medical implant capable of being replaced by naturally ingrown tissue over time upon implantation. The assembly or joint assembling the two separately manufactured electrospun heart valve components together is an ultrasonic weld or a glued weld.

In one embodiment, the tissue engineered heart valve is structured such that the two separately manufactured electrospun heart valve components are not sewn or stitched together, or the two separately manufactured electrospun heart valve components do not have any sewn areas or stitches.

In a further embodiment, the tissue engineered heart valve has a support structure assembled or joint together in between the two separately manufactured electrospun heart valve components.

The present invention further provides a method of assembling two separately manufactured electrospun components to form a cardiovascular medical implant or medical implant. A first component is electrospun using a type of biodegradable electrospun polymers. Separately from the first component, a second component is electrospun using the same type of biodegradable electrospun polymers as in the first component. The first electrospun component and the second electrospun component are then assembled by the same type biodegradable electrospun polymers as used in the first electrospun component and the second electrospun component creating an assembled medical implant that is a porous, biodegradable medical implant capable of being replaced by naturally ingrown tissue over time upon implantation. Examples of assembling is ultrasonic welding, gluing or glue welding. Examples of assembled cardiovascular medical implants or medical implants are where the first electrospun component and the second electrospun component are components of a tissue engineered heart valve, a tissue engineered vascular graft, or a tissue engineered vessel.

In one embodiment, the method excludes sewing or stitching such that the first electrospun component and the second electrospun component are not sewn or stitched together, or the first electrospun component and the second electrospun component do not have any sewn areas or stitches.

In another embodiment, the method includes a further step of assembling a support structure in between the first electrospun component and the second electrospun component. Examples of such support structures are a stent, a frame, a braided structure or a mesh structure to support the cardiovascular medical implant or medical implant. In the example of making such a device the first electrospun component is electrospun, then the support structure is applied over the first electrospun component after which the second electrospun component is electrospun over the support structure and the first electrospun component. The assembling or joining (e.g. via ultrasonic welding) then connects the first and the second electrospun components, therewith laminating the support structure therebetween.

In yet another embodiment, the step of assembling includes patterning an area where the first electrospun component and the second electrospun component are assembled or joint together. In one variation of the embodiments, the assembly or joining (e.g. ultrasonic welding) could be performed in a substantially circumferential pattern, a helical pattern or a circular pattern, which in one embodiment could improve kink resistance of a medical implant.

Embodiment of the invention have the following advantages, which for a medical implant concept like a heart valve is that two electrospun components of the heart valve avoids the use of sutures, which then omits the drawbacks of sutures such as stress concentrations, manual processing, etc., while keeping potential of ETR because microstructure/porosity is only impacted locally, if even, at the interface between the materials. Tissue can form inside scaffold around local weld. Assembly or joining electrospun components according to this invention will be more precise, reproducible and could be automated compared to suturing or sewing. Furthermore, assembly or joining electrospun components according to this invention avoids the need for extra materials such as sutures or other fixatives as it uses the same polymer as the electrospun structure is composed of.

Embodiment of the invention have the following advantages, which for a medical implant concept like vascular graft or vessel concept are the same as for the heart valve. In addition, the mere fact of avoiding sutures avoids suture holes, which improves hemostasis (bleeding) of the graft or vessel upon implantation. Furthermore, a patterned weld allows for tuning/optimization of mechanical properties, which in one example improves kink resistance. In one aspect, an ultrasonic welding pattern itself could become the support structure that ensures sufficient kink resistance.

### BRIEF DESCRIPTION OF THE DRAWINGS

FIG. 1 shows according to an exemplary embodiment of the invention a first electrospun component 110 having a type of biodegradable electrospun polymers, a second electrospun component 120 having the same type of biodegradable electrospun polymers as in the first electrospun component 110, In one example, the second electrospun component is separately manufactured from the first electrospun component as portrayed in the top aspect of FIG. 1. The bottom aspect of FIG. 1 shows the first electrospun component 110 and the second electrospun component 120 assembled or joint together by either an ultrasonic weld or a glue weld (both 130) by the same type biodegradable electrospun polymers as in the first electrospun component and the second electrospun component. The assembly forms a medical implant capable of undergoing endogenous tissue regeneration (ETR).

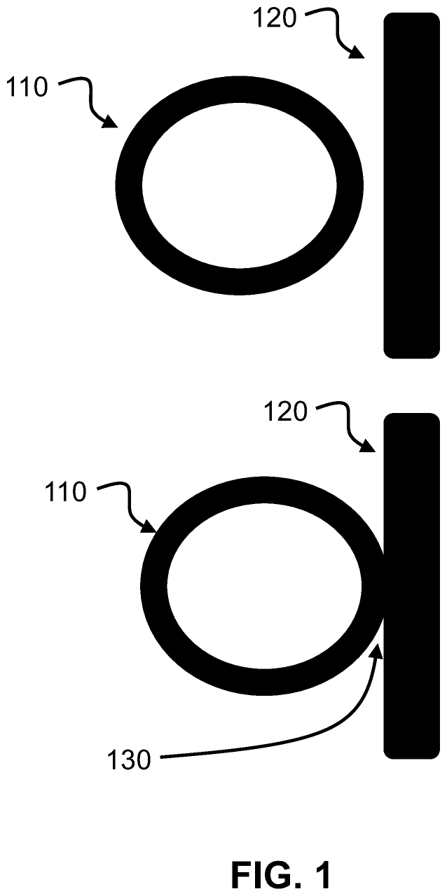

FIG. 2 shows in a cross section view according to an exemplary embodiment of the invention in the upper left panel a configuration of a circular polymeric tube cover and a sheet polymeric leaflet before being welded together, in the upper right panel a configuration of tube cover and polymeric leaflet being welded together ultrasonically. This picture shows the cross section of the upper view at the middle of the welded surface, and in the bottom panel a zoom into welded cross section where a solid attachment can be seen between the two components, while the mesh morphology of the polymeric tube cover and the polymeric leaflet is still visible where the welding device touched the polymeric pieces. The solid attachment is seen clearly deeper between the components where the change of morphology is not so relevant for ETR.

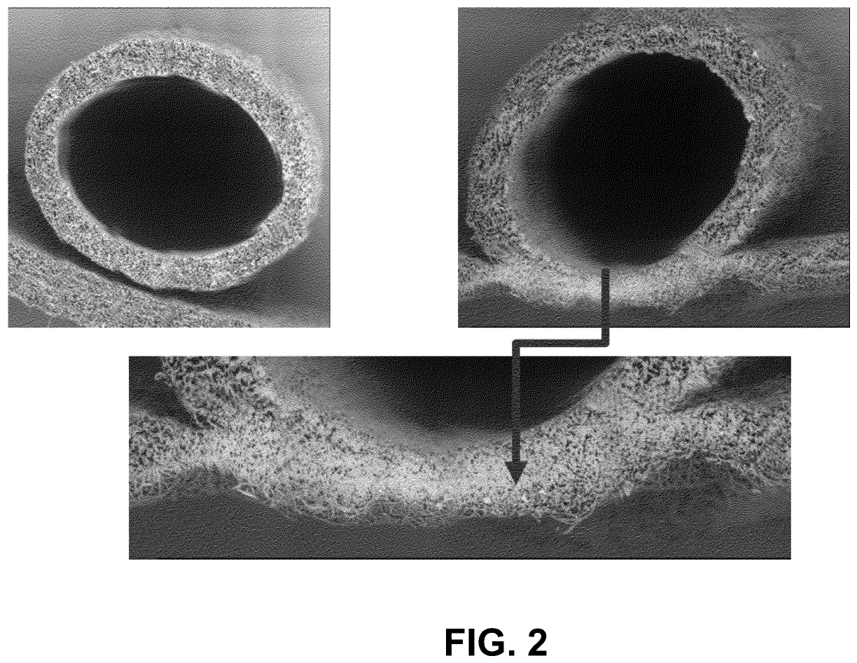

FIG. 3 shows in a cross section view according to an exemplary embodiment of the invention in the left panel a cross section view of a tube cover welded with a leaflet in a fully assembled aortic valve (SEM picture), in the middle panel a zoom in of image in the left panel clearly showing that the outside area maintains its mesh morphology while the solid weld is created on the inside of the connection between the components, and in the right panel a side view of a welded area where the welding device has touched the leaflet (along the direction of the arrow). One can see a vertical seam mark along the weld line while the mesh morphology is maintained almost fully. This is the foundation to an unchanged morphology on the outside of the device and therefore to a successful ETR of the device.

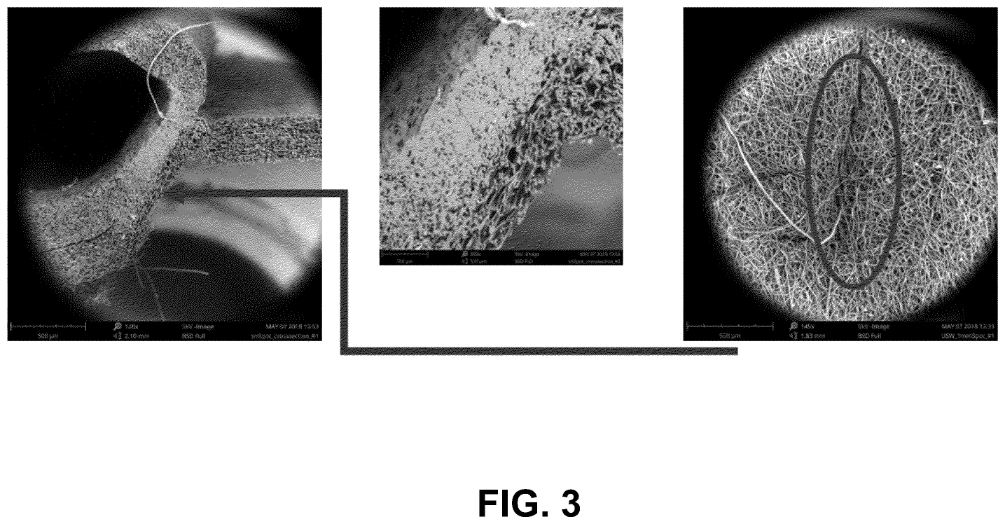

FIG. 4 shows an example laser cut material according to an exemplary embodiment of the invention example production steps of welding concept (steps 1 to 5—see text).

FIG. 5 shows in a section view and according to an exemplary embodiment of the invention glued posts observed under SEM

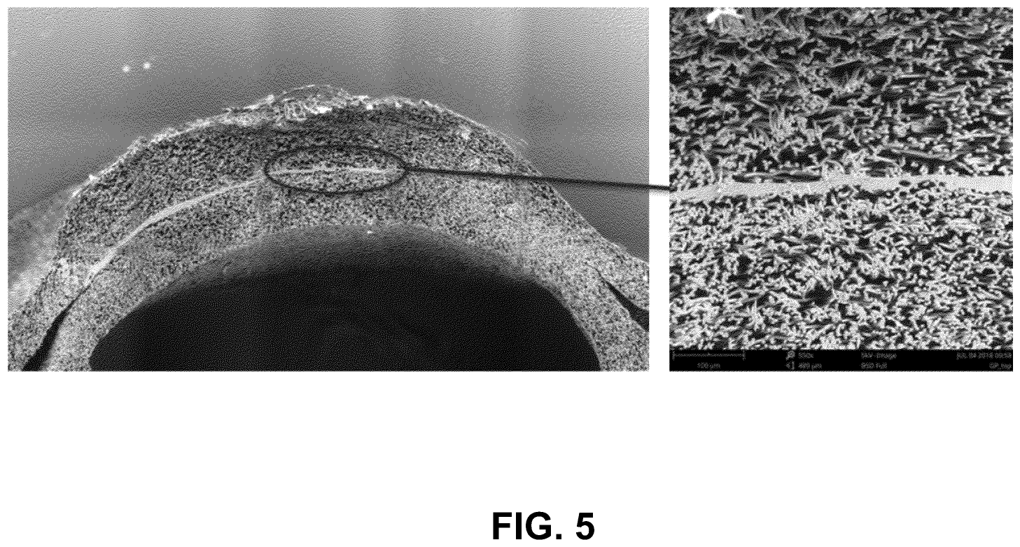

FIG. 6 shows in a section and side view and according to an exemplary embodiment of the invention ultrasonic welding “wedge” configuration.

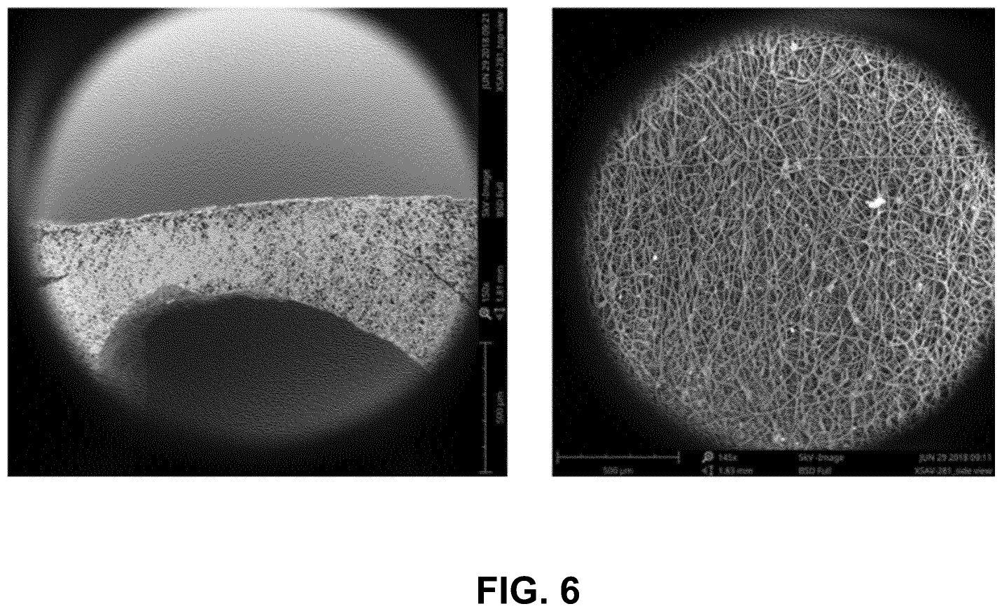

### DETAILED DESCRIPTION

Embodiments of the invention are medical implants formed by separate components which are made of the same electrospun polymers and whereby the separate components are assembled or joint together using their own polymers or the same type of polymers. Two examples are provided. In the first example, the two separate components are ultrasonically welded together such that the polymers of the two separate components create the assembly or joint. In the second example, the two separate components are glued together with the same polymers as used for the separate polymers. In other words, the medical implant is composed of two separately created components which are assembled or joint together using the same polymers as used for the creation of the components. This results in an implant having the same polymers yet individually created. An example of such implant is heart valve with one or more leaflet components and possibly one or more support structures which is then assembled without sewing or stitching. The present invention is not limited to this example as it would apply to any type of electrospun implant where components are individually manufactured yet assembled together to form a medical implant. Important to realize is that despite the assembly or joint as a result of the welding or gluing, the medical implants resulting from this assembly retain their structural features in terms of being porous, as well as being biodegradable and/or having the ability to undergo ETR once implanted in a body.

In other embodiments of the invention, medical implants with support structures are formed by components which are made of the same electrospun polymers and whereby the components are assembled or joint together using their own polymers or the same type of polymers, yet laminating the support structure in between the two electrospun components. Examples of such support structures are a stent, a frame, a braided structure or a mesh structure to support the cardiovascular medical implant or medical implant. In the example of making such a device the first electrospun component is electrospun, then the support structure is applied over the first electrospun component after which the second electrospun component is electrospun over the support structure and the first electrospun component. The assembling or joining (e.g. via ultrasonic welding) then connects the first and the second electrospun components, therewith laminating the support structure therebetween.

### Ultrasonic Welding

Ultrasonic welding is an industrial technique whereby high-frequency ultrasonic acoustic vibrations are locally applied to workpieces being held together under pressure to create a solid-state weld. Ultrasonic welding of thermoplastics causes local melting of the plastic due to heat generated by friction. Ultrasonic welding can be used for all kind of polymers in theory.

The process of ultrasonic welding includes various parameters that have to be determined for specific applications to yield optimal weld quality. Such as frequency, amplitude, welding energy, time and pressure. There is also a need for full process development and optimization including dedicated tools (e.g. sonotrodes and boosters) to allow different welding shapes and sizes. There is not an “off the shelf solution” available for each and every material.

The known benefit of ultrasonic welding is that it is much faster than conventional adhesives or solvents. The drying time is very quick, and the pieces do not need to remain in a jig for long periods of time waiting for the joint to dry or cure. The welding can easily be fully automated, making clean and precise joints—the weld is considered to be very clean and reproducible and rarely requires any touch-up work. The low thermal impact on the materials involved enables a greater number of materials to be welded together.

In the medical industry ultrasonic welding is considered for use because it does not introduce contaminants or degradation into the weld and the process can be specialized for use in clean rooms. The highly automated process provides strict control over dimensional tolerances and does not interfere with the biocompatibility of the materials used.

Nevertheless, nothing is known about the change of surface morphology during welding and therefore the characteristics with regard to biodegradation and ETR.

In fact, a skilled artisan would refrain from using welding since the polymer is in the end “melted” and small polymer fibers would melt, thereby losing the specific set-up and mechanical properties as well as morphology of the surface will change.

For the specific application in the heart where a porous mesh of polymeric fibers is exposed to the blood flow and rapid tissue growth is desired as part of the ETR process, it is crucial that the mechanical properties and the morphology of the mesh should be maintained for optimal cell ingrowth. For the purposes of this invention, we found that macroscopically, welding does not change the surface characteristics of porous polymer structures, therefore the structure still provides excellent ETR characteristics. In addition, in this invention it was found that the created weld was surprisingly durable which is essential for the medical implant application.

In one example, stitches on posts for heart valve are replaced by using ultrasonic welding. Preferably, any welded segments on the implantable device should not be fully exposed to biological flow of blood cells. Since the local melted region of such a segment is potentially originated between two layers of polymers, hidden from the outer surface, this technology is of high potential.

Preferably, only one polymer is used for the production of the valve and additional materials like suture material can be avoided. This is especially important since the whole device will show same degradation rates.

Another example of utilizing ultrasonic welding for a medical application is via a vascular graft application, where two electrospun tubular layers can be joint together in one or more desired locations by a desired pattern. The welding pattern can be utilized for embedding a support structure in between the two electrospun layer for specific mechanical properties (i.e. a strain relief structure) without reduced risk of the electrospun layers delaminating from one another. A welding pattern can also be used to affect the mechanical properties of the graft, e.g. helical pattern of a continuous weld can help a graft to bend without it kinking.

The ultrasonic welding method used for the embodiments herein can be characterized with the following parameters used during the process. A welding frequency between 45-70 kHz could be used, whereby a welding frequency of around 70 kHz is preferred. A welding energy of 0.1-5 W*sec could be applied, whereby a welding energy of 0.3-1.5 W*sec is preferred. Regarding the welding time 0.1-5 sec could be used, whereby a welding time of 0.1-2 sec is preferred.

The weld created according to the method of this invention was examined under SEM where one can see that the welded area is stamped via a horn on the outer side of the leaflet. Although the weld is initiated via a contact of a horn on the outer leaflet surface, the mesh morphology (e.g. porosity and fiber diameter) remains surprisingly the same within the welded outer surface area. Therefore, it was concluded that this welding process can be used successfully for ETR applications. Since the outer surface will not block or reduce the ETR effect, it might even actually improve the effect as it allows elimination of foreign objects (e.g. stitching wires) and heat effected zones (e.g. laser cut holes for stitching wires) which are potentially a source for calcification deposits and reduced ETR around these locations.

During the welding process a certain frequency is generated in which small mechanical vibrations are transmitted via several possible “sonotrodes” or horns to two polymeric pieces that should be attached to each other. The horn is in touch with the outside of one first polymeric piece and is designed to deliver a specific amplification (gain) of these vibrations to the polymer. A rapid frictional energy is generating heat locally between the inner side of the first polymeric piece and the outer side of the second polymeric piece. Thereby the outside layer of the second polymeric piece is shaped in a way that allows it to be stretched on a metallic frame at the post of the surgical valve. Preferably the metallic frame is used as an anvil to apply the initial contact surface between the polymer pieces. Together with the pressure applied via the horn on the outside layer of the first polymeric piece, this contact is crucial for a local accurate weld. Since the metallic frame of an artificial surgical heart valve is normally made of titanium or titanium alloy, it is preferred that the material of the horn is also titanium.

The heat generated via the aforementioned frictional energy will result in a local melting pattern that will create a strong attachment between the two polymeric pieces. Surprisingly this attachment has shown to be strong enough to endure in vitro accelerated wear testing conditions sufficiently. In addition, lower deflection of the posts during full loading of the valve was observed repeatedly. This is explained by having a less-rigid attachment between the leaflets and the frame (than in state-of-the-art stitched configuration) allowing the stiff frame posts to resist bending better, hence- to deflect less. The connection between low post deflection and improved durability can indicate lower stress concentrations at the posts.

This pattern will also allow ETR to occur in vivo, mainly due to the fact that the outer surface of the polymer that is exposed to blood flow will maintain its material properties in terms of porosity, mesh morphology, etc.

When stitches are used, you need high forces to keep the valve in the correct position. Sutures are normally strong and rigid and not flexible or yielding. Therefore, the leaflets are lashed down in a specific position and little movements are not possible. We have identified that it is very important to give the final valve some possibility to “settle in” a good position and adjust to the perfect shape. By using welded seams instead of sutures, we allow the final valve to find the best position and thereby relieve the whole structure from high stress points that lead to tearing and failure of the device. In addition, the seams do not contain additional material (like sutures) so the whole system can more or less move in a uniform way.

Preferably, the welding area or horn can be soaked with fluid and/or water. Since the melting point of the polymer is below 100° C., an essential attribute which dictates the water will not evaporate but allow generation of specific weld patterns that further creates a morphology that is supporting ETR as well as providing sufficient durability of the weld. A person skilled in the art would therefore refrain from welding wet polymer pieces together since it is expected to damage the uniformity of the weld or even not to weld at all.

The assembly between leaflet and frame other than in the posts could also be made via ultrasonic welding between the bottom of the polymeric leaflet and the metallic frame PET cover.

According to the embodiments provided herein, the problems related to stitches are eliminated and heart valve ETR compatibility and hemodynamic performance are improved. Another notable advantage in replacing stitches with ultrasonic welding is the rapid assembly time with welding the posts. Where usually, accurate stitches locations are being sewed slowly and carefully, welding instead will require about 75% less assembly time. Eventually, this allows more capability to assemble valves rapidly for fast iterations during process development stage and later on for cost effectiveness of a final commercial product.

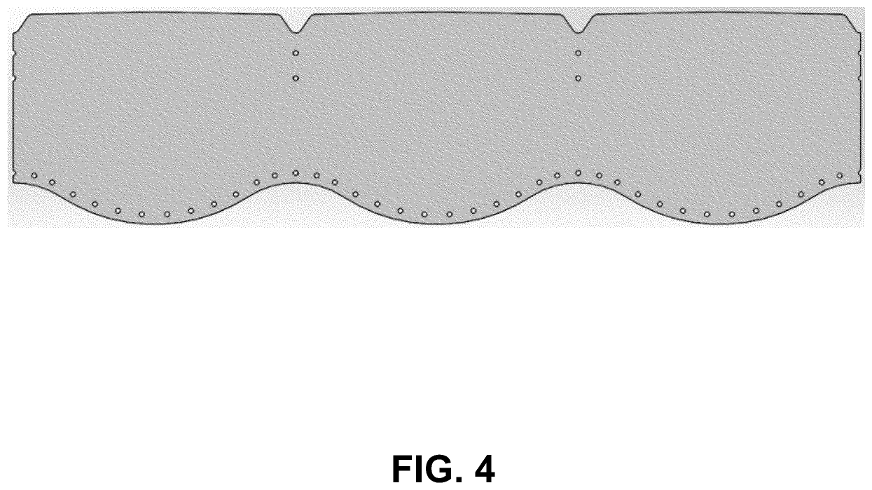

An example of production steps of welding concept is as follows.
Step 1: Spinning is performed as usual, but with a dedicated drawing with only two laser-cut stitching holes at each post (for fixation stitches). Drawing can possibly include laser engraved marks for more accurate welding. Stitching holes can also be replaced by similar engraved marks. It is noted that when using welding instead of a running stitch at the bottom of the leaflet, the bottom laser cut holes are not cut as shown in FIG. 4.
Step 2: The valve is assembled partially—with a full running stitch at the bottom of the frame and only a single fixation stitch at each post. The running stitch can also be replaced by a running weld over the circumference as it has been shown that a PET fabric surprisingly welds well resulting in a strong attachment.
Step 3: The leaflets are welded ultrasonically to the post covers (tubes) on the sides of each post while forcing the leaflets in a closed configuration—two welds for each post. An alternative configuration allows a similar weld (at both sides of post) within a single weld-touch instead of two.
Step 4 (not mandatory): The fixation stitches are removed from the middle of each post. This step is not mandatory as for several configurations, the fixation stitch is kept since it is not located in the dynamic part of the leaflet and does allow additional strength in attachment of leaflet to post besides the weld.
Step 5 (not mandatory): At the position where the fixation stitch was removed, a second welding step takes place (at the middle of each post). This step is not mandatory, other configurations are applicable, including one where the middle of the post remains unwelded due to sufficient strength of weld even without it.
Step 6: The final assembled valve is where the leaflets are only connected by welds. This specific configuration demonstrates the replacement of a running stitch at the bottom of the frame with a running weld. Therefore, this picture in FIG. 4 shows a valve free of stitches according to the invention.

These methods steps could be varied depending on the desired goal and structure that is intended to be made, and the invention is not limited to these steps or this order of steps.

### Gluing

Gluing is not considered for the connection of e.g. valves due to known disadvantages:
Adhesive compounds that are needed for the gluing have (depending on their chemical basis) a limited thermal and chemical resistance or load-bearing capacity. Therefore, the mechanical properties of the bond are temperature-dependent and different compared to the properties of the bonded material. This will lead to areas of higher stress and less durability.
The long-term stability of a bond is subject to aging processes and will therefore change during the life-time of a device which is not acceptable for a medical device.
For the gluing process adhesives and auxiliary compounds are typically needed that are not biocompatible or even toxic and/or need specific precautions.

In most commercial tissue valves there is a stiffness difference between the tissue used for the covering and the metallic frame that often causes abrasion and may lead to failure of the device. Therefore, different components are used to protect the posts, which are usually made from a softer material than the frame (e.g.—synthetic fabric or plastic liners). Still, there is a need for improvement and further development.

A typical embodiment of a surgical aortic valve includes three tubular components made of the same polymer which are used as post covers. These tubes (post covers) are located on the valve frame and their purpose is to decrease abrasion between the dynamic leaflet and the static metallic frame.

Embodiments of this invention leverage on these abrasion-protective components made from an electrospun material produced from the polymer since an attachment is created in the form of gluing between the leaflets scaffolds and the posts instead of using stitches.

Gluing takes place by using a solution of the same polymer and applying the solution on the area where attachment is needed between the leaflets and the frame posts. The viscous solution is brushed locally on the surface of the post covers and the leaflet scaffold is spun directly upon it while the solution is still in the liquid state. When the solution dries and solidifies, it creates solid attachment between the leaflet scaffold and the post covers. This solid polymeric layer creating the attachment is not exposed to blood flow so ETR can take place effectively. Moreover, this configuration has no stitches at all around the posts and therefore suggests even faster tissue growth than is seen in the state-of-the-art configuration.

While using the same polymer for spinning as well as for gluing the bond has similar material properties as the bonded material. This can potentially lead to a better stress distribution. Pressure points can move and this finally creates higher durability.

Relevant for a good fatigue endurance of an artificial valve frame is the deflection of the frame posts or the inwards bending of the posts. Too high of a post deflection can also lead to poor leaflet function and excessive pin-wheeling. When applying this new gluing method for attachment of the leaflets the posts can resist the deflection more easily. This is then resulting in a lower post deflection what can be measured when doing post deflection measurements.

Surprisingly it was found by the inventors that a valve produced according to the current invention shows low sensitivity to unequal volume in the attachment region between the posts with regard to durability—both in vitro and in vivo.

An additional important advantage in the proposed invention is that the assembly process of the scaffold after the gluing process is significantly faster than state of the art configurations. It takes approximately four hours for the state-of-the-art configuration while the proposed configuration suggests a much faster assembly of approximately one hour.

A further advantage of the configuration is the possible elimination of the lower stitches. Besides the solid attachment at the posts, there is only one circumferential running stitch at the bottom of the leaflets to the base of the metallic frame. The bottom of all posts is free of stitches, which allows the polymeric scaffold to set its shape when loaded and allow optimal pressure distribution. This results in elimination of high stress concentration points around the posts and therefore improves valve durability.

An example production steps of glued posts concept is:
Step 1: Assemble the spinning target.
Step 2: Attach a polymeric post cover made from a polymer in each post. Then assemble again the target.
Step 3: Secure the target into the spinning box and manually brush each post completely with wet polymer solution.
Step 4: Spin the scaffold and remove it from spinning box when done.
Step 5: If desired, cut the scaffold according to a valve design pattern.
Step 6: Assemble the valve on its designated frame without any attachment at the posts except for the tubes stretched over top of the frame.
Step 7: Conclude assembly with a running stitch at the bottom of each leaflet.

These methods steps could be varied depending on the desired goal and structure that is intended to be made, and the invention is not limited to these steps or this order of steps.

One more notable method for production of a glued posts concept is to electro spin the leaflet and the post covers separately and then to attach these components together using a dedicated jig. A leaflet is held expanded in an accurate position until post covers with an accurate amount of glue are inserted in desired location and leaflet is released with applied pressure locally at the posts until glue is fully dry. In this method, it is easier to assure equal volume of glue at each post and to eliminate potential variation in bonds surface between posts thus, potentially improving valve durability.

Definition of Polymer for the Purposes of this Invention

The polymers or supramolecular polymers referenced in this document may comprise the ureido-pyrimidinone (UPy) quadruple hydrogen-bonding motif (pioneered by Sijbesma (1997), Science 278, 1601-1604) and a polymer backbone, for example selected from the group of biodegradable polyesters, polyurethanes, polycarbonates, poly(orthoesters), polyphosphoesters, polyanhydrides, polyphosphazenes, polyhydroxyalkanoates, polyvinylalcohol, polypropylenefumarate. Examples of polyesters are polycaprolactone, poly(L-lactide), poly(DL-lactide), poly(valerolactone), polyglycolide, polydioxanone, and their copolyesters. Examples of polycarbonates are poly(trimethylenecarbonate), poly(dimethyltrimethylenecarbonate), poly(hexamethylene carbonate).

The same result may be obtained with alternative, non-supramolecular polymers, if properties are carefully selected and material processed to ensure required surface characteristics. These polymers may comprise biodegradable or non-biodegradable polyesters, polyurethanes, polycarbonates, poly(orthoesters), polyphosphoesters, polyanhydrides, polyphosphazenes, polyhydroxyalkanoates, polyvinylalcohol, polypropylenefumarate. Examples of polyesters are polycaprolactone, poly(L-lactide), poly(DL-lactide), poly(valerolactone), polyglycolide, polydioxanone, and their copolyesters. Examples of polycarbonates are poly(trimethylenecarbonate), poly(dimethyltrimethylenecarbonate), poly(hexamethylene carbonate).

What is claimed is:

1. A cardiovascular medical implant, comprising:
(a) a first electrospun component having a type of biodegradable electrospun polymers; and
(b) a second electrospun component having the same type of biodegradable electrospun polymers as in the first electrospun component,
wherein the first electrospun component and the second electrospun component are assembled or joint together by the same type biodegradable electrospun polymers as in the first electrospun component and the second electrospun component, and
wherein the assembled cardiovascular medical implant is a porous, biodegradable medical implant capable of being replaced by naturally ingrown tissue over time upon implantation.

2. The cardiovascular medical implant as set forth in claim 1, wherein the assembly or joint assembling the first electrospun component and the second electrospun component together is an ultrasonic weld or is a glued weld.

3. The cardiovascular medical implant as set forth in claim 1, wherein the first electrospun component and the second electrospun component are components of a tissue engineered heart valve, a tissue engineered vascular graft, or a tissue engineered vessel.

4. The cardiovascular medical implant as set forth in claim 1, wherein the first electrospun component and the second electrospun component are not sewn or stitched together, or the first electrospun component and the second electrospun component do not have any sewn areas or stitches.

5. The cardiovascular medical implant as set forth in claim 1, further comprising a support structure assembled or joint together in between the first electrospun component and the second electrospun component.

6. The cardiovascular medical implant as set forth in claim 1, wherein the second electrospun component is separately manufactured from the first electrospun component.

7. A tissue engineered heart valve, comprising: two separately manufactured electrospun heart valve components, each manufactured with the same type of biodegradable electrospun polymers, wherein the two separately manufactured electrospun heart valve components are assembled or joint together by the same type biodegradable electrospun polymers, wherein the assembled tissue engineered heart valve is a porous, biodegradable medical implant capable of being replaced by naturally ingrown tissue over time upon implantation.

8. The tissue engineered heart valve as set forth in claim 7, wherein the assembly or joint assembling the two separately manufactured electrospun heart valve components together is an ultrasonic weld or a glued weld.

9. The tissue engineered heart valve as set forth in claim 7, wherein the two separately manufactured electrospun heart valve components are not sewn or stitched together, or the two separately manufactured electrospun heart valve components do not have any sewn areas or stitches.

10. The tissue engineered heart valve as set forth in claim 7, further comprising a support structure assembled or joint together in between the two separately manufactured electrospun heart valve components.

11. A method of assembling two separately manufactured electrospun components to form a cardiovascular medical implant, comprising:
(a) electrospinning a first component using a type of biodegradable electrospun polymers;
(b) electrospinning a second component using the same type of biodegradable electrospun polymers as in the first component; and
(c) assembling the first electrospun component and the second electrospun component by the same type biodegradable electrospun polymers as used in the first electrospun component and the second electrospun component,
wherein the assembled cardiovascular medical implant is a porous, biodegradable medical implant capable of being replaced by naturally ingrown tissue over time upon implantation.

12. The method as set forth in claim 11, wherein the assembling is ultrasonic welding, gluing or glue welding.

13. The method as set forth in claim 11, wherein the first electrospun component and the second electrospun component are components of a tissue engineered heart valve, a tissue engineered vascular graft, or a tissue engineered vessel.

14. The method as set forth in claim 11, wherein the first electrospun component and the second electrospun component are not sewn or stitched together, or the first electrospun component and the second electrospun component do not have any sewn areas or stitches.

15. The method as set forth in claim 11, further comprising assembling a support structure in between the first electrospun component and the second electrospun component.

16. The method as set forth in claim 11, wherein the assembling step further comprises patterning an area where the first electrospun component and the second electrospun component are assembled or joint together.

17. The method as set forth in claim 11, wherein the second component is electrospun separately from the first component.
